SEQUENCE LISTING (4 sequences)

SEQ ID NO: 1 - DNA, 31 bp, Artificial
  PCR primer
gctccatggcctaatattacaaacttgtgcc

SEQ ID NO: 2 - DNA, 29 bp, Artificial
  PCR primer
tgctctagactcaagtgtctgtggatcac

SEQ ID NO: 3 - DNA, 21 bp, Artificial
  PCR primer
caatggtttaacaggcacagg

SEQ ID NO: 4 - DNA, 21 bp, Artificial
  PCR primer
ctcaagtgtctgtggatcacg

(19) United States
(12) Patent Application Publication
(10) Pub. No.: US 2023/0241037 A1
(43) Pub. Date: Aug. 3, 2023

(54) CROSS-LINKED MEDICATION FOR TREATMENT OF CORONAVIRAL INFECTION AND METHOD OF TREATMENT

(71) Applicant: 1GLOBE HEALTH INSTITUTE, NANJING, Nanjing (CN)

(72) Inventor: Chiang J. LI, Cambridge, MA (US)

(21) Appl. No.: 17/910,779

(22) PCT Filed: May 10, 2021
(86) PCT No.: PCT/CN2021/092693

§ 371 Date: Nov. 9, 2022

(30) Foreign Application Priority Data
Mar. 9, 2020 (CN) 202010158032.X

(51) Int. Cl. A61K 31/426; A61K 31/7056; A61P 31/14

(52) U.S. Cl. CPC A61K 31/426; A61K 31/7056; A61P 31/14

(57) ABSTRACT

The present invention provides a combinatorial drug comprising nitazoxanide or tizoxanide and ribavirin, and the use thereof for treating or preventing coronavirus infection. The combinatorial drugs provided are particularly effective in the treatment of diseases caused by SARS-CoV-2 (COVID-19).

### FIELD OF THE INVENTION

The present invention relates to combinatorial drugs and treatment methods for treating or preventing coronavirus infection, especially acute respiratory infectious diseases or diseases of which pneumonia is a major symptom, both kinds of diseases caused by SARS-CoV-2 (Novel Coronavirus) infection.

### REFERENCE TO A SEQUENCE LISTING

Sequence listings and related materials in the ASCII text file named “GHI028_seq_ST25.txt” and created on Feb. 8, 2023, with a size of about 1 kilobyte, is hereby incorporated by reference.

### BACKGROUND OF THE INVENTION

The acute respiratory infectious disease (COVID-19), which is caused by SARS-CoV-2, has posed an unprecedented threat to the lives and health of human beings since its global outbreak at the end of 2019. SARS-CoV-2 belongs to the same subfamily of coronaviruses as SARSr-CoV and MERSr-CoV, but the degree of genomic homology between them is not high. The new type of pneumonia caused by SARS-CoV-2 infection is highly contagious, and difficult to control; human populations are generally susceptible to such infections, which has become a long-term problem. Although drug research and development against SARS-CoV-2 has been vigorously promoted globally, there is not any particularly effective therapeutic drug, and there is still an urgent clinical need to find a therapeutic solution that can effectively treat or prevent the disease caused by its infection.

Nitazoxanide (NTZ) is a safe, orally bioavailable thiazolide, approved in the United States of America for treatment of Cryptosporidium parvum and Giardia lamblia infections, which is part of the treatment of transmissible gastroenteritis. This drug has been used in many countries, such as those in Africa and South America, and the results show that it is effective against parasitic infection without adverse reactions. Tizoxanide (TIZ) is the main active metabolite of nitazoxanide in vivo. Nitazoxanide and tizoxanide were originally used as drugs to treat protozoal infections, and in later studies they have been found to affect the activity of cellular inflammation-related proteins including COX-2 (cyclooxygenase), NF-κB, p65, IκBα, ERK1/2 and p38MAPK, and therefore used to treat inflammatory-related diseases, including lipopolysaccharide (LPS) and other bacterial endotoxin-induced inflammatory diseases. Another study revealed that nitazoxanide has an inhibitory effect on the activity of acetyl-CoA carboxylase in HepG2 cells, and can have the effect of reducing obesity, lowering blood lipid level, preventing fatty liver and preventing atherosclerosis (CN110478357A). In addition, some studies have suggested that nitazoxanide and tizoxanide can reduce the duration of symptoms associated with human respiratory syncytial virus (CN108289961A), and have potentially effects on clinic treatment of rotavirus, norovirus, HBV and HCV infections (Jean-François Rossignol, Romark Laboratories, L. C., 2014; CN108289961A); however, there is no data to support its antiviral activity in vivo. Nitazoxanide-based mechanism against viral infections is not yet clear.

Ribavirin is an antiviral, nucleoside analogue agent, approved by the U.S. Food and Drug Administration (FDA) for the treatment of hepatitis C, viral hemorrhagic fever, and severe lower respiratory tract infection caused by respiratory syncytial virus (RSV) (aerosol inhalation only). Although it has been shown to have inhibitory effects on a variety of other DNA and RNA viruses (such as herpes simplex virus, influenza virus, etc.) in vitro, its indications are generally limited due to lack of definitive clinical evidence. FDA clearly indicated in the approved drug label that ribavirin is not suitable for the treatment of influenza et al. (COPEGUS® (ribavirin) Tablets, Initial U.S. Approval: 2002). Ribavirin, alone or in combination with systemic glucocorticoids, has been tried for empiric treatment of severe acute respiratory syndrome (SARS) caused by SARS-CoV infection, but its clinical benefit has been disappointing (WHO. Management of severe acute respiratory syndrome (SARS) 2003 Apr. 11).

### SUMMARY OF THE INVENTION

The present invention provides a combination drug regimen for treating or preventing coronavirus infections, especially those caused by the 2019 novel coronavirus (SARS-CoV-2), specifically, for inhibiting coronavirus infections, alleviating or reversing disease progression where pneumonia is the main symptom as caused by coronavirus infections, or preventing coronavirus infections.

In one aspect, the present invention relates to a novel combinatorial pharmaceutical and its use in the treatment or prevention of coronavirus infections, or its associated diseases or conditions. Where relevant, the infections or the associated diseases refer to the diseases or conditions caused by SARS-CoV-2, SARS-CoV, MERS-CoV, 229E, NL63, OC43 and/or HKU1 infections, especially SARS-CoV-2 infections and pneumonia caused thereby.

In one embodiment, the combinatorial pharmaceutical comprises: at least one pharmaceutical selected from the group consisting of nitazoxanide, tizoxanide, and a pharmaceutically acceptable salt thereof; and at least one pharmaceutical selected from the group consisting of ribavirin, a prodrug, metabolite, derivative, enantiomer, diastereomer, tautomer, or racemate thereof, and any pharmaceutically acceptable salt or ester of the foregoing.

In one embodiment, the combinatorial pharmaceutical comprises at least one pharmaceutical selected from nitazoxanide and a pharmaceutically acceptable salt thereof, and at least one pharmaceutical selected from ribavirin and a pharmaceutically acceptable salt or ester thereof.

In one feature, the nitazoxanide is administered in an oral formulation, including as a tablet, suspension, or capsule.

In one feature, the ribavirin can be administered in an oral formulation, an inhalation formulation or an injection formulation. In one embodiment, ribavirin is administered in an aerosol inhalation formulation directly via the respiratory tract.

In one embodiment, the combinatorial pharmaceuticals disclosed by the present invention are used to treat, reduce or alleviate at least one symptom of above-described diseases or conditions associated thereof, where the symptoms comprise at least one selected from the group consisting of cough, decreased appetite, asthma, dyspnea, fever, malaise, dizziness, nausea, diminished or lost sense of smell, and pneumonia.

In one embodiment, the use of the combinatorial drugs disclosed by the present invention is for the preparation of a pharmaceutical composition for the treatment or prevention of coronavirus infection or diseases or conditions associated thereof, where the coronavirus is SARS-CoV-2, SARS-CoV, MERS-CoV, 229E, NL63, OC43 and/or HKU1, especially SARS-CoV-2, where the associated diseases or conditions is pneumonia.

In another aspect, the present invention relates to a method for treating or preventing coronavirus infection, or a disease or a condition associated thereof, where the method comprises administering a therapeutically effective amount of the novel combinatorial pharmaceuticals mentioned above to a subject in need. The associated diseases or conditions are acute respiratory infectious disease or pneumonia.

In one embodiment, the treatment or prevention method comprises administering to a subject in need thereof: a therapeutically effective amount of at least one pharmaceutical selected from the group consisting of nitazoxanide, tizoxanide and a pharmaceutically acceptable salt, or any solvate thereof; and a therapeutically effective amount of at least one pharmaceutical selected from the group consisting of ribavirin, a prodrug, metabolite, derivative, enantiomer, diastereomer, tautomer, or racemate thereof, and any pharmaceutically acceptable salt or ester of the foregoing.

In one embodiment, the treatment or prevention method comprises administering to a subject in need thereof a therapeutically effective amount of at least one pharmaceutical selected from nitazoxanide and a pharmaceutically acceptable salt thereof, and a therapeutically effective amount of at least one pharmaceutical selected from ribavirin and a pharmaceutically acceptable salt or ester thereof.

In some embodiments, nitazoxanide or tizoxanide can be administered to the subject simultaneously or sequentially with ribavirin.

In one feature, the nitazoxanide is administered in an oral formulation, including as a tablet, suspension, or capsule.

In one feature, the ribavirin can be administered in an oral formulation, an inhalation formulation or an injection formulation. In one embodiment, ribavirin is administered in an aerosol inhalation formulation directly via the respiratory tract.

In one feature, the subject is a severely ill patient whose sickness was caused by coronavirus infection, and ribavirin is administered in an aerosol inhalation formulation directly via the respiratory tract.

In one embodiment, the coronavirus is SARS-CoV-2, SARS-CoV, MERS-CoV, 229E, NL63, OC43 and/or HKU1, especially SARS-CoV-2, and the associated disease or condition is pneumonia.

In some embodiments, the method of preventing a disease or condition caused by or associated with coronavirus infection refers to chemoprevention/drug prevention.

In some embodiments, the administration can be used to prevent progression from mild to severe illness in a patient, or to alleviate at least one symptom selected from the group consisting of cough, decreased appetite, asthma, dyspnea, fever, malaise, dizziness, nausea, reduced or lost sense of smell, and pneumonia.

The foregoing and other objects, aspects, features and advantages of the present invention are further apparent from the following description and the claims.

### BRIEF DESCRIPTION OF FIGURES

FIG. 1 illustrates the results of cytotoxicity of nitazoxanide (NTZ), ribavirin (RBV) and GH23 (NTZ+RBV) in Vero E6 cells tested by the standard CCK-8 assay.

FIG. 2 illustrates the antiviral activities of NTZ, RBV, and GH23 anti SARS-CoV-2 in vitro.

### DETAILED DESCRIPTION OF THE INVENTION

### I. Definition

As used in the description and claims, the singular form “a”, “an”, or “the” includes plural references, unless the context clearly dictates otherwise. For example, the term “a cell” includes a plurality of cells including mixtures thereof.

As used herein, “about” is used herein to modify a numerical value above and below the stated value by a variance of 10%. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%”.

Unless dictates otherwise, all of the technical and scientific terms used herein have the same meaning as is generally understood by a person of ordinary skill in the art to which the present invention belongs.

The term “effective amount” of an active agent refers to the amount that is sufficient to elicit the required biological response. As understood by those of ordinary skill in the art, the effective amount of a compound of the present invention is vary depending on such factors as the desired biological endpoints, the pharmacokinetics of the compound, the disease being treated, the mode of administration, and the patient.

The terms “treatment”, “treating” or “therapy” (of) a disease or disorder refer to a method of reducing, delaying or improving such a condition before or after it occurs. Treatment may be directed at one or more effects or symptoms of the disease and/or the underlying pathology. The treatment can be any reduction and can be, but is not limited to, the complete eliminate of the disease or symptoms of the disease. As compared with an equivalent untreated control, such degree of reduction or prevention as measured by any standard technique is at least 5%, 10%, 20%, 40%, 50%, 60%, 80%, 90%, 95%, or 100%.

As used herein, the term “subject” refers to any animal (e.g., a mammal), including, but not limited to humans, non-human primates, rodents, and the like, which is to be the recipient of a particular treatment or prevention scheme. Typically, the terms “subject,” “patient,” and “individual(s) in need of prophylactic protection from infection or population(s) at high risk” are used interchangeably herein and refer to human subject(s).

The term “and/or” should be understood to mean “either or both” of the elements conjoined, i.e., elements that are conjunctively present in some instances and disjunctively present in some other instances. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. When a range of values is listed herein, it is intended to encompass each value and sub-range within that range. For example, “1-5 mg” is intended to encompass 1 mg, 2 mg, 3 mg, 4 mg, 5 mg, 1-2 mg, 1-3 mg, 1-4 mg, 1-5 mg, 2-3 mg, 2-4 mg, 2-5 mg, 3-4 mg, 3-5 mg, and 4-5 mg.

The terms “administer”, “administering”, or “administration” are used herein in their broadest sense. These terms refer to any method of introducing to a subject a compound or combinatorial pharmaceutical described herein, and can include, for example, introducing the compound systemically, locally, or in situ to a subject. Thus, a compound of the present disclosure produced in a subject from a combinatorial drug (whether or not it includes the compound) is encompassed in these terms. When these terms are used in connection with the term “systemic” or “systemically”, they generally refer to in vivo systemic absorption or accumulation of the compound or combinatorial drug in the blood stream followed by distribution throughout the entire body.

“Composition” or “combinatorial (combination)” therapy or treatment as used herein refers to the administration of at least two different drugs for the treatment of a disorder, condition or symptom, for example, viral pneumonia, viral infection, coronaviral pneumonia and the like. Such combination therapy comprises administering one drug before, at the time of, and/or after the administration of the other drug. The interval period between administrations of the different drugs is up to several weeks, but is more commonly within 48 hours, and most commonly within 24 hours.

The compounds of the present invention may form salts which are also within the scope of this invention. Reference to a compound of the present invention herein is understood to include reference to salts thereof, unless otherwise indicated. The term “salt(s)”, as employed herein, denotes acidic and/or basic salts formed with inorganic and/or organic acids and bases. In addition, when the compound of the present invention contains both a basic moiety (such as, but not limited to, a pyridine or imidazole) and an acidic moiety (such as, but not limited to, a carboxylic acid), zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation.

“Chemoprevention” as used herein refers to the administration of the combinatorial drug regimen provided herein to asymptomatic persons with a viral infection, or to persons whose viral infection test is negative but are considered to have a possibility of viral infection by epidemiologic history analysis, or persons whose viral infection test is negative but are considered to have a viral infection like pneumonia by other medical diagnosis (such as medical imaging analysis, etc.), as prophylactic measures, to prevent or mitigate the pathogenicity of the viral infection.

The term “synergy”, “synergistic”, “synergistically” or “enhanced” as used herein refers to an effect of interaction or combination of two or more components to produce a combined effect greater than the sum of their separate effects (or “additive effects”).

### II. Combinatorial Drugs, Uses and Treatment Methods Thereof

The present invention provides a novel combinatorial pharmaceutical and its use in the treatment or prevention of coronavirus infection (particularly, SARS-CoV-2 infection) or associated diseases or conditions thereof (such as pneumonia, particularly, COVID-19). The present invention also provides methods for treating or preventing a coronavirus infection (particularly, a SARS-CoV-2 infection), or associated diseases or conditions thereof (such as pneumonia, particularly, COVID-19), comprising administering to a subject in need of a therapeutically effective amount of the combinatorial drug described herein.

The novel combinatorial drug provided by the present invention, in one embodiment, comprises: at least one thiazolide compound selected from the group consisting of nitazoxanide, tizoxanide or derivatives thereof, and any pharmaceutically acceptable salt of the foregoing or any solvate of the foregoing; and at least one compound selected from the group consisting of a ribavirin, a prodrug, metabolite, derivative, enantiomer, diastereomer, tautomer, or racemate thereof, and any pharmaceutically acceptable salt or ester of the foregoing.

The present invention also provides the use of the combinatorial drug in the preparation of pharmaceutical compositions for the treatment or prevention of coronavirus infection or associated diseases or conditions thereof, where the coronavirus includes, but is not limited to, SARS-CoV-2, SARS-CoV, MERS-CoV, 229E, NL63, OC43 or HKU1, especially SARS-CoV-2, where the associated diseases or conditions thereof is acute respiratory infectious diseases, including pneumonia.

The chemical name of Nitazoxanide is as follows: o-[N-(5-nitro-1,3-thiazol-2-yl)carbamoyl]phenol acetate); its chemical formula is as follows:

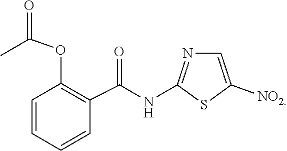 [SMILES: CC(=O)Oc1ccccc1C(=O)Nc1ncc([N+](=O)[O-])s1]

The chemical name of Tizoxanide is: 2-Hydroxy-N-(5-nitro-2-thiazolyl)benzamide; its chemical formula is as follows:

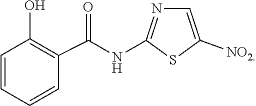 [SMILES: O=C(Nc1ncc([N+](=O)[O-])s1)c1ccccc1O]

The chemical name of Ribavirin is: 1-[(2R, 3R, 4S, 5R)-3,4-dihydroxy-5-(hydroxymethyl) oxolan-2-yl]-1H-1,2,4-triazole-3-carboxamide, also known as 1-(β-D-ribofuranosyl)-1H-1,2,4-triazole-3-carboxamide, with CAS registration number 36791-04-5; its chemical formula is as follows:

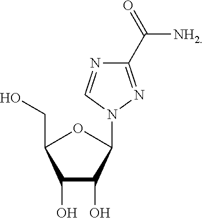

The present invention provides nitazoxanide or tizoxanide in combination with ribavirin for synergistic inhibition of coronavirus infections, such as SARS-CoV-2 infection.

The above findings provide available combination therapy regimen for the treatment of SARS-CoV-2 or other coronavirus infections and associated diseases or conditions thereof, such as pneumonia, that can achieve effective treatment of the coronavirus infection, or rapid alleviation of conditions associated thereof, or reduction in the duration of clinical symptoms, as well as diminishing the potential for coronavirus infection and retransmission.

In one embodiment, the combinatorial drugs disclosed by the present invention show no new adverse events and have similar safety profiles compared to each drug of the combinatorial drugs when used alone.

In one embodiment, the combinatorial drug disclosed by the present invention can prevent the progression from mild to severe illness, or alleviate at least one symptom, where the symptom comprises at least one selected from the group consisting of cough, decreased appetite, asthma, dyspnea, fever, malaise, dizziness, nausea, diminished or lost sense of smell, and pneumonia.

In another aspect, the present invention relates to a method for treating or preventing coronavirus infection or associated diseases or conditions thereof. The method comprises administering to a subject in need thereof a therapeutically effective amount of at least one thiazolide compound, selected from nitazoxanide, tizoxanide or derivatives thereof, a pharmaceutically acceptable salt of any of the foregoing, and any solvate of the foregoing; and a therapeutically effective amount of a compound selected from at least one ribavirin, a prodrug, metabolite, derivative, enantiomer, diastereomer, tautomer, or racemate thereof, and any pharmaceutically acceptable salt or ester of the foregoing.

In one embodiment, the coronavirus is SARS-CoV-2, SARS-CoV, MERS-CoV, 229E, NL63, OC43 or HKU1, preferably SARS-CoV-2. In one embodiment, the associated diseases or conditions thereof is pneumonia.

In one embodiment, the method comprises administering to a subject in need thereof a therapeutically effective amount of nitazoxanide, and a therapeutically effective amount of ribavirin.

In some embodiments, nitazoxanide and ribavirin are administered to the patient simultaneously, contemporaneously, separately, and/or sequentially.

In some embodiments, nitazoxanide is administered to a subject at a total daily dose of about 100 mg to about 2000 mg. In some embodiments, nitazoxanide is administered once daily (QD), twice daily (BID), three times daily (TID), or four times daily (QID).

In some embodiments, nitazoxanide is administered to a subject in a single dose of about 100 mg to about 1000 mg. In some embodiments, nitazoxanide is administered to a subject in a single dose selected from the group consisting of about 100 mg, about 150 mg, about 200 mg, about 250 mg, about 300 mg, about 350 mg, about 400 mg, about 450 mg, about 500 mg, about 550 mg, about 700 mg, about 800 mg, about 900 mg, and about 1000 mg.

In some embodiments, nitazoxanide is administered to subjects in a single dose from about 300 mg to about 500 mg, twice daily (BID) or three times daily (TID).

In some embodiments, nitazoxanide is administered to subjects in a dose selected from the group consisting of about 200 mg BID, about 250 mg BID, about 300 mg BID, about 350 mg BID, about 400 mg BID, about 450 mg BID, about 500 mg BID, about 550 mg BID, about 600 mg BID, about 200 mg TID, about 250 mg TID, about 300 mg TID, about 350 mg TID, about 400 mg TID, about 450 mg TID, about 500 mg TID, about 550 mg TID, about 600 mg TID, about 200 mg QID, about 250 mg QID, about 300 mg QID, about 350 mg QID, about 400 mg QID, about 450 mg QID, and about 500 mg QID.

In some embodiments, nitazoxanide is administered to a subject in a dose of about 500 mg BID.

In some embodiments, nitazoxanide is administered to a subject in a dose of about 300 mg to about 350 mg TID.

In some embodiments, ribavirin is administered to a subject in a total daily dose of about 20 mg to about 1200 mg. In some embodiments, nitazoxanide is administered once daily (QD), twice daily (BID), or three times daily (TID).

In one embodiment, ribavirin is administered to a subject in a single dose of about 20 mg to about 400 mg.

In some embodiments, nitazoxanide is administered to a subject in a single dose selected from the group consisting of about 20 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, and about 500 mg.

In some embodiments, ribavirin is administered to a subject in a single dose of about 50 mg to about 400 mg, twice daily (BID) or three times daily (TID).

In some embodiments, ribavirin is administered twice daily (BID). In some embodiments, ribavirin is administered to a subject in a dose selected from the group consisting of about 20 mg BID, about 50 mg BID, about 60 mg BID, about 80 mg BID, about 100 mg BID, about 120 mg BID, about 140 mg BID, about 150 mg BID, about 160 mg BID, about 180 mg BID, about 200 mg BID, about 220 mg BID, about 240 mg BID, about 250 mg BID, about 260 mg BID, about 280 mg BID, about 300 mg BID, about 320 mg BID, about 330 mg BID, about 340 mg BID, about 350 mg BID, about 360 mg BID, about 380 mg BID, about 400 mg BID, about 420 mg BID, about 440 mg BID, about 450 mg BID, about 460 mg BID, about 480 mg BID and about 500 mg BID. In some embodiments, ribavirin is administered to a subject in a dose of about 200 mg BID. In some embodiments, ribavirin is administered to a subject in a dose of about 400 mg BID.

In some embodiments, ribavirin is administered to a subject three times daily (TID). In some embodiments, ribavirin is administered to a subject in a dose selected from the group consisting of 20 mg TID, about 50 mg TID, about 60 mg TID, about 80 mg TID, about 100 mg TID, about 120 mg TID, about 140 mg TID, about 150 mg TID, about 160 mg TID, about 180 mg TID, about 200 mg TID, about 220 mg TID, about 240 mg TID, about 250 mg TID, about 260 mg TID, about 280 mg TID, about 300 mg TID, about 320 mg TID, about 330 mg TID, about 340 mg TID, about 350 mg TID, about 360 mg TID, about 380 mg TID and about 400 mg TID. In some embodiments, ribavirin is administered to a subject at a dose of about 100 mg TID. In some embodiments, ribavirin is administered to a subject in a dose of about 150 mg TID.

In some embodiments, nitazoxanide and ribavirin can be administered simultaneously for the first dose. In some embodiments, the interval period between the first administration of nitazoxanide and ribavirin ranges from about 0 hours to about 16 hours. In some embodiments, the interval period between the first administration of nitazoxanide and ribavirin is at least 0 hour, at least 0.1 hour, at least 0.2 hour, at least 0.2 hour, at least 0.3 hour, at least 0.4 hour, at least 1 hour, at least 2 hours, at least 3 hours, at least 4 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours and/or at least 16 hours.

In some embodiments, nitazoxanide and ribavirin are administered to a subject twice daily (BID), respectively. Nitazoxanide and ribavirin may be administered simultaneously for the first dose or the interval period between the first administration of nitazoxanide and ribavirin ranges from about 0 hour to about 8 hours, where nitazoxanide is administered in a single dose of about 300 mg to about 500 mg, and ribavirin is administered in a single dose of about 200 mg to about 400 mg.

In some embodiments, nitazoxanide and ribavirin are administered to a subject three times a day (TID), respectively. Nitazoxanide and ribavirin may be administered simultaneously for the first dose or the interval period between the first administration of nitazoxanide and ribavirin ranges from about 0 hour to about 4 hours, wherein nitazoxanide is administered in a single dose of about 200 mg to about 350 mg, wherein ribavirin is administered in a single dose of about 100 mg to about 200 mg.

In some embodiments, the method of preventing a disease or a condition caused by or associated with coronavirus infection refers to chemoprevention/drug prevention.

In one embodiment, the administration may be used to prevent progression from mild to severe illness, or to alleviate at least one symptom selected from the group consisting of cough, decreased appetite, asthma, dyspnea, fever, malaise, dizziness, nausea, diminished or lost sense of smell, and pneumonia.

### III. Examples

### Example 1 Cell Preparation

Vero E6 African green monkey kidney cell line was obtained from American Type Culture Collection (ATCC, No. 1586) and maintained in MEM medium (MEM; Gibco Invitrogen) supplemented with 10% fetal bovine serum (FBS; Gibco Invitrogen) at 37° C. in an atmosphere of 5% CO₂.

### Example 2 Isolation of Virus Strain

The SARS-CoV-2 (strain HB-01) was obtained from the China Centers for Disease Control and Prevention (China CDC). The complete genomic sequence for the SARS-CoV-2 has been submitted to GISAID (BetaCoV/Wuhan/IVDC-HB-01/2020|EPI_ISL_402119), and deposited in China National Microbiological Data Center (accession No. NMDC10013001, genome accession No. MDC60013002-01). The virus was propagated in Vero E6 cells, the virus titer was determined by 50% tissue culture infective dose (TCID₅₀) by immunofluorescence assay. All infection experiments were performed in a Biosafety Level 3 (BLS-3) laboratory.

### Example 3 Drug Candidates

Ribavirin (RBV; Cat No.: R101754) and nitazoxanide (NTZ; Cat No.: N159057) were purchased from Aladdin. GH23 is a combinatorial drug of NTZ and RBV.

### Example 4 Viral RNA Extraction and Quantitative Analysis (qRT-PCR)

SARS-CoV-2 viral RNA was extracted with MiniBEST viral RNA/DNA Extraction Kit (Takara, Cat No.: 9766), the specific operations were performed according to the manufacturer's instructions.

Reverse transcription was performed using a PrimeScript RT Reagent Kit with gDNA Eraser (Takara, Cat No.: RR047A); qRT-PCR test was performed using a StepOnePlus™ Real-time PCR system (Applied Biosystem) with TB Green Premix Ex Taq II (Takara, Cat No.: RR820A).

SARS-CoV-2 viral RNA was extracted from 100 μL of cell culture supernatant, and was eluted at 30 μL of ribonuclease free water, and 3 μL of total RNA was first digested with gDNA Eraser to remove contaminating genomic DNA, and the first strand cDNA was synthesized in 20 μL reaction volume. Then cDNA (2 μL) was used as a template for PCR. The receptor binding domain (RBD) of the spike gene was amplified from the cDNA template by PCR using the following primers:

RBD-Forward:
(SEQ ID NO: 1)
5′-GCTCCATGGCCTAATATTACAAACTTGTGCC-3′;
RBD-Reverse:
(SEQ ID NO: 2)
5′-TGCTCTAGACTCAAGTGTCTGTGGATCAC-3′.

The amplified PCR product was cloned into pMT/BiP/V5-His vector (Invitrogen), and used as the plasmid standard after its identity was confirmed by sequencing. A standard curve was generated by determination of copy numbers from serial dilutions (103-109 copies) of the plasmid. The primers used for quantitative real-time PCR were:

RBD-qF1:
(SEQ ID NO: 3)
5′-CAATGGTTTAACAGGCACAGG-3′;
RBD-qR1:
(SEQ ID NO: 4)
5′-CTCAAGTGTCTGTGGATCACG-3′.

PCR amplification was performed as follows: denaturation at 95° C. for 5 minutes, and then proceeding with 40 cycles including three steps: 95° C. for 15 seconds, 54° C. for 15 seconds, and 72° C. for 30 seconds.

### Example 5 Cytotoxicity Test of Drug Candidates

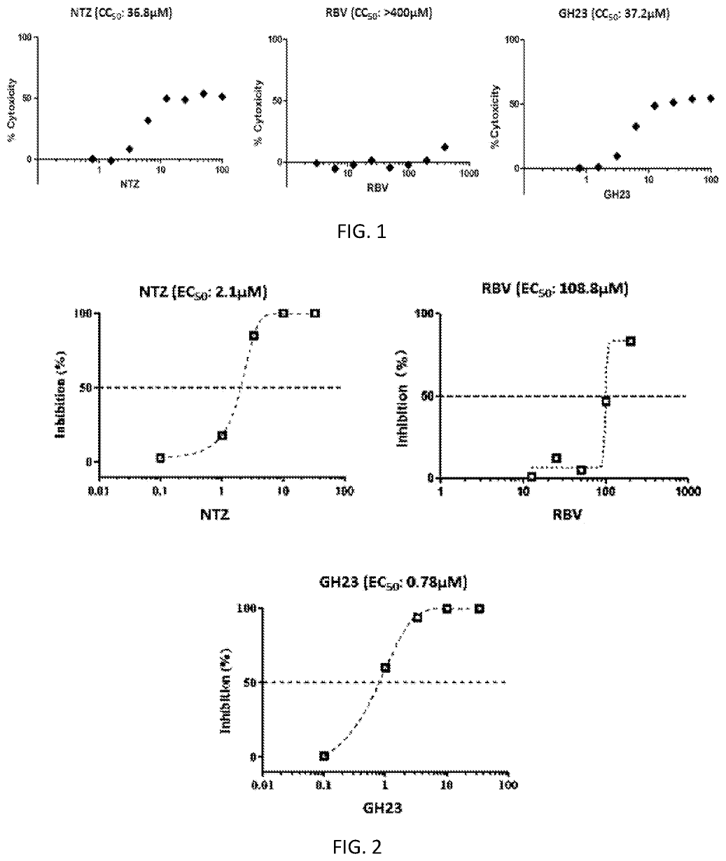

Standard CCK-8 assays were performed to measure the cytotoxic effect of the drug candidates on Vero E6 cells, which was performed in accordance with the manufacturer's instructions (Beyotime, China). In these studies, the tested drug (NTZ, RBV, GH23) was incubated with Vero E6 cells for 48 hours. The analysis results of standard CCK8 are shown in FIG. 1.

Incubation of Vero E6 cells with NTZ resulted in dose-dependent cytotoxicity with a CC₅₀ of 36.8 μM. By contrast, no cytotoxic effect was observed when Vero E6 cells were treated with RBV (CC₅₀>400 μM). The CC₅₀ value for GH23 (NTZ+RBV) was 37.2 μM, which was essentially the same as that seen with NTZ alone. This result suggests that the combination of NTZ and RBV does not have synergistic toxicity.

### Example 6 Antiviral Activity Test of Drug Candidates

To assess the antiviral efficacy of the combinatorial drugs according to the present invention, Vero E6 cells were cultured overnight in 48 wells cell culture petri dish with a density of 1×10⁴ cells/well. Cells were pretreated with different concentrations of test compounds for 1 hour. DMSO was used as a negative control. The virus (MOI of 0.05) was subsequently added to infect cells for 2 hours. The virus-drug mixture was then removed, and the cells were further cultured with fresh test drug-containing medium. At 48 hours of post viral infection, the cell supernatants were collected and lysed in lysis buffer (Takara, Cat No. 9766) for quantitative analysis of viral RNA levels. The dose-response curves of viral RNA copies versus the drug concentrations were plotted using GraphPad Prism 6 software, to evaluate the ability of each drug for inhibiting SARS-CoV-2 viral replication.

The results are shown in FIG. 2. Treatment of Vero 6 cells with NTZ alone (0-30 μM) resulted in inhibition of SARS-CoV-2 levels with an EC₅₀ of 2.1 μM, whereas the EC₅₀ value for treatment of Vero 6 cells with RBV alone was 108.8 μM. This finding is consistent with a prior study which found that while ribavirin is significantly incorporated into coronavirus RNA it is also readily excised by the 3′-5′ exoribonuclease (ExoN) activity of coronavirus protein nsp14 (Ferrona F et al. PNAS. 2017 115(2):E162-E171). Combined treatment of Vero 6 cells with GH23 (NTZ and RBV), however, resulted in significant synergistic inhibition of SARS-CoV-2 levels with an EC₅₀ of 0.78 μM.

A summary of the CC₅₀, EC₅₀ and corresponding selectivity index (SI) values for NTZ, RBV and GH23 are shown in Table 1. The selectivity indices for NTZ, RBV and GH23 were 17.5, >3.7 and 47.7, respectively. These data suggest that combined use of NTZ and RBV has a higher safety window for the treatment of patients suffering from SARS-CoV-2 than NTZ or RBV monotherapy.

TABLE 1
antiviral activity of GH23 against SARS-CoV-2

| Drugs | CC₅₀ (μM) | EC₅₀ (μM) | SI |
|---|---|---|---|
| NTZ | 36.8 | 2.1 | 17.5 |
| RBV | >400 | 108.8 | >3.7 |
| GH23 (NTZ + RBV) | 37.2 | 0.78 | 47.7 |

1. A combinatorial drug, comprising: a first compound selected from the group consisting of nitazoxanide, tizoxanide, and a pharmaceutically acceptable salt thereof; and a second compound selected from the group consisting of a ribavirin, and a pharmaceutically acceptable salt or ester thereof.

2. A method of using the combinatorial drug of claim 1 in the preparation of a pharmaceutical composition for treating or preventing a disease or a condition caused by coronavirus infection.

3. The method of claim 2, wherein the coronavirus is selected from the group consisting of SARS-CoV-2, SARS-CoV, MERS-CoV, 229E, NL63, OC43 and HKU1.

4. The method of claim 2, wherein the disease or the condition caused by the coronavirus infection is an acute respiratory infectious disease or pneumonia.

5. The method of claim 2, wherein the coronavirus is SARS-CoV-2, and the disease or the condition caused by the coronavirus infection is a disease caused by the SARS-CoV-2 infection (COVID-19).

6. The combinatorial drug of claim 1, wherein the first compound is in an oral formulation.

7. The combinatorial drug of claim 1, wherein the second compound is in an oral formulation, an inhalation formulation or an injection formulation.

8. A method for treating or preventing a disease or condition caused by or associated with coronavirus infection, the method comprising administering to a subject in need thereof a therapeutically effective amount of at least a first compound selected from the group consisting of nitazoxanide, tizoxanide, and a pharmaceutically acceptable salt thereof, as well as a therapeutically effective amount of at least a second compound selected from the group consisting of a ribavirin, and a pharmaceutically acceptable salt or ester thereof, wherein the first compound and the second compound are administered to the subject simultaneously or sequentially.

9. The method of claim 8, wherein the coronavirus is selected from the group consisting of SARS-CoV-2, SARS-CoV, MERS-CoV, 229E, NL63, OC43 and HKU1.

10. The method of claim 8, wherein the disease or the condition caused by the coronavirus infection is an acute respiratory infectious disease or pneumonia caused by SARS-CoV-2 infection.

11. The method of claim 8, wherein the method is used to prevent progression from mild to severe illness, or to alleviate at least one symptom selected from the group consisting of cough, decreased appetite, asthma, dyspnea, fever, malaise, dizziness, nausea, diminished or lost sense of smell, and pneumonia.

12. The method of claim 8, wherein the first compound is administered in an oral formulation.

13. The method of claim 8, wherein the second compound is administered in an oral formulation, an inhalation formulation or an injection formulation.

14. The method of claim 8, wherein the subject is a patient with severe illness caused by coronavirus infection, and the second compound is administered to the subject in an inhalation formulation.

15. The method of claim 8, m wherein the first compound is administered to the subject in a total daily dose of about 100 mg to about 2000 mg, and the second compound is administered to the subject in a total daily dose of about 20 mg to about 1200 mg, and wherein the first and second compounds are administered twice a day (BID) or three times a day (TID), respectively.

16. The method of claim 15, wherein the second compound is administered to the subject in a single dose of about 100 mg to about 600 mg.

17. The method of claim 15, wherein the second compound is administered to the subject in a single dose of about 20 mg to about 400 mg.

18. The method of claim 8, wherein the method for preventing a disease or a condition caused by or associated with coronavirus infection further comprises a chemoprevention method or a drug prevention method.
